(19) United States
(12) Patent Application Publication
(10) Pub. No.: US 2022/0347195 A1
(43) Pub. Date: Nov. 3, 2022

(54) PSYCHEDELICS FOR TREATMENT OF PAIN

(71) Applicant: Mind Medicine, Inc., New York, NY (US)

(72) Inventors: Robert BARROW, Madison, WI (US); Daniel R KARLIN, New York, NY (US)

(73) Assignee: Mind Medicine, Inc., New York, NY (US)

(21) Appl. No.: 17/732,878

(22) Filed: Apr. 29, 2022

Related U.S. Application Data
(60) Provisional application No. 63/183,578, filed on May 3, 2021.

(51) Int. Cl. A61K 31/675; A61K 31/48; A61K 31/4045; A61K 31/137; A61P 25/04

(52) U.S. Cl. CPC A61K 31/675; A61K 31/48; A61K 31/4045; A61K 31/137; A61P 25/04

(57) ABSTRACT

A method of treating pain, by administering an effective amount of a psychedelic to an individual and treating pain in the individual.

### BACKGROUND OF THE INVENTION

### 1. Technical Field

The present invention relates to compositions and methods for treating pain. More specifically, the present invention relates to the use of psychedelics in treating pain.

### 2. Background Art

People feel pain when nerves detect tissue or nerve damage and/or real or perceived bodily harm and transmit information about the damage to the brain. Acute pain is usually temporary and felt with an injury or surgery and treating the injury can relieve the pain. Chronic pain can last much longer than acute pain (weeks, months, or years), and can be continuous or intermittent and stopping for periods of time. Examples of chronic pain include arthritis, migraine headaches, cancer pain, phantom limb pain, back pain, pain from fibromyalgia and nerve pain. Chronic pain can also cause emotional effects in an individual, such as depression, anger, anxiety, and fear of re-injury.

Just as chronic pain can make an individual feel in a bad mood, their mood can also actually cause pain or making preexisting pain increase, known as psychogenic pain. Psychogenic pain can be caused by pain memory (the nervous system remembers pain after an injury has healed) or by signals in the brain being confused with physical pain. Anxiety, bipolar disorder, depression, and stress can all contribute to the feeling of physical pain.

Pain is currently treated with either non-steroidal anti-inflammatory drugs (NSAIDS), opioids or antidepressants such as selective serotonin reuptake inhibitors (SSRIs). NSAIDS such as aspirin, ibuprofen, ketoprofen, naproxen work by blocking Cox-1 and Cox-2 enzymes which help make prostaglandins in the body. Prostaglandins are released by damaged tissue and increase the feeling of pain, so by reducing the amount of prostaglandins, less pain is felt. Using NSAIDs regularly to treat pain can lead to ulcers in the esophagus, stomach, and small intestine, cause damage to the kidneys, as well as increased risk of heart attacks and stroke. Opioids produce pain relief as well as euphoria by binding and activating opioid receptors on nerve cells involved in feeling pain. When attached to receptors, opioids block signals from the brain and release dopamine in the body. Opioids include oxycodone hydrochloride (OXYCONTIN® (Purdue Pharma)), hydrocodone and acetameniophen (VICODIN® (AbbVie)), oxycodone and acetominaphen (PERCOCET® (Endo Pharmaceuticals), among others. Opioid overuse can lead to addiction.

Psychedelics are substances capable of inducing exceptional subjective effects such as a dream-like alteration of consciousness, affective changes, enhanced introspective abilities, visual imagery, pseudo-hallucinations, synesthesia, altered temporal and special perception, mystical-type experiences, disembodiment, and ego dissolution (Liechti, 2017; Passie, Halpern, Stichtenoth, Emrich & Hintzen, 2008).

Psychedelics can be used to assist psychotherapy for many indications including anxiety, depression, addiction, personality disorder and others and can also be used to treat other disorders such as cluster headache and migraine and others (Passie et al., 2008; Hintzen et al., 2010; Nichols, 2016; Liechti, 2017). Psilocybin has been useful in treating depression and anxiety. Johnson, et al. (Potential Therapeutic Effects of Psilocybin. Neurotherapeutics (2017) 14:734-740 (Jun. 5, 2017)) states that with mood and anxiety disorders, three controlled trials have suggested that psilocybin may decrease symptoms of depression and anxiety in the context of cancer-related psychiatric distress for at least six months following a single acute administration.

Castellanos, et al. (Chronic pain and psychedelics: a review and proposed mechanism of action. Regional Anesthesia & Pain Medicine 2020;45:486-494) states that several studies and reports over the past 50 years have shown potential analgesic benefit for use of psychedelics in cancer pain, phantom limb pain, and cluster headache. While the mechanisms by which the classic psychedelics may provide analgesia are not clear, several possibilities exist given the similarity between 5-HT_{2A} activation pathways of psychedelics and the nociceptive modulation pathways in humans. Additionally, the alterations in FC seen with psychedelic use suggest a way that these agents could help reverse the changes in neural connections seen in chronic pain states.

Gerard (Pain, Death, and LSD: A Retrospective of the Work of Dr. Eric Kast) describes the work of Dr. Kast in comparing duration of analgesia produced by narcotics meperidine, dihydromorphinone, and LSD in grave and terminally ill patients. It was found that LSD produced greater and longer lasting pain relief than the narcotics following acute treatment (e.g., one or two doses); however following treatment with LSD, pain returned after several days. It was proposed that LSD provided analgesic relief due to 1. “(LSD) seems to deprive the patient of his ability to concentrate on one specific sensory input, even if the input is of urgent survival value.”; 2. “. . . ‘minor’ sensations, namely those of less importance for survival, make a claim on the patient's attention sometimes in preference to those of major survival significance.”; 3. “(LSD) diminishes cortical control of thoughts, concepts, or ideas and reduces their significance in control of vegetative function and behavior in general. The meaning of pain . . . and its frightful resonance . . . is greatly alleviated.”; and 4. “ . . . LSD obliterates the individual's ego boundaries (and) a geographic separation can more easily be made between the self and the ailing part.”

Ramaekers, et al. (Journal of Psychopharmacology, 2021, Vol. 35(4) 398-405) examined the use of LSD as an analgesic at dose levels not expected to produce profound mind-altering effects. Doses of 5, 10, and 20 micrograms of LSD were administered, and a Cold Pressor Test was performed to assess pain tolerance. The 20 microgram dose significantly increased the time that participants could tolerate cold water exposure and decreased subjective levels of experienced pain and unpleasantness.

There is an unmet need for providing pain relief in patients. Patients in areas of oncology, neuropathy, and other areas find far below minimal pain relief while becoming opiate addicts. Karra, et al. (Future Medicine, Pain Management, Vol. 11, No. 3) describes that general practitioners do not have sufficient knowledge, time, or resources to properly manage patients who have chronic pain. There are not enough specialists in pain to treat patients. Varassi, et al. (Curr Med Res Opin. 2010 May;26(5):1231-45) describes that chronic pain management is inadequate due to poor communication between patients and physicians, side effects of analgesic drugs, and limits on individualized therapy.

Therefore, there remains a need for effective methods of treating pain that avoid the unwanted side effects of NSAIDS, opioids or selective serotonin reuptake inhibitors.

### SUMMARY OF THE INVENTION

The present invention provides for a method of treating pain, by administering an effective amount of a psychedelic to an individual and treating pain in the individual.

### DETAILED DESCRIPTION OF THE INVENTION

The present invention provides for a method of treating pain, by administering an effective amount of a psychedelic to an individual and treating pain in the individual.

“Pain” as used herein can refer to any discomfort in the body. The pain can be the general types of acute (such as injury or paper cut), chronic, nociceptive (such as post-surgical pain, visceral, somatic, or radicular), neuropathic, inflammatory, or functional. Chronic pain can be further classified as chronic primary pain (characterized by disability or emotional distress and not better accounted for by another diagnosis of chronic pain) or chronic secondary pain (such as chronic cancer-related pain, chronic post surgical or post-traumatic pain, chronic neuropathic pain, chronic secondary headache or orofacial pain, chronic secondary visceral pain, or chronic secondary musculoskeletal pain).

The pain can be caused from a physical state in the body (such as injury, damaged tissue, surgery, cancer or cancer breakthrough, diabetes, migraines or other headaches, arthritis, fibromyalgia, back pain, nerve pain, shingles, radiation, or chemotherapy drugs) as well as an emotional state (such as anxiety or depression).

The psychedelics in the present invention can be, but are not limited to, lysergic acid diethylamide (LSD), psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, solvates thereof, isomers thereof, analogs thereof, or homologues thereof. Preferably, the dose of the psychedelic is one that provides a meaningful clinical effect or can be a dose that is a perceptual dose or subperceptual dose. The psychedelic drug can be administered as a single dose or as repeat doses over multiple days, weeks, months, or years. A dose of 0.05-1 mg (10-1000 μg) can be used of LSD. Psilocybin can be dosed at 1-50 mg, psilocin can be dosed at 1-100 mg, mescaline can be dosed at 10-1000 mg, 5-MeO-DMT can be dosed at 0.2-20 mg, DMT can be dosed at 10-100 mg, DOI can be dosed at 0.1-10 mg, and DOB can be dosed at 0.1-5 mg. Effects of a single dose of the psychedelic drug can last 1-12 hours after administration, and the individual can be supervised by medical personnel such as a psychiatrist during this time. If lower doses are given, medical supervision can be unnecessary.

Mechanistically, psychedelics act as nonspecific serotonin agonists. LSD potently stimulates the 5-HT_{2A} receptor but also 5-HT_{2B/C}, 5-HT₁ and D₁₋₃ receptors (Rickli et al., 2016). Serotonergic psychedelics have their psychoactive/hallucinogenic effects by agonism at the serotonin 5-HT_{2A} receptor. LSD induces its psychedelic effects in humans primarily via stimulation of the 5-HT_{2A} receptor (Kraehenmann et al., 2017; Preller et al., 2017; Barrett et al., 2018). Psilocybin (3-(2-dimethylaminoethyl)-1H-indol-4-yl] dihydrogen phosphate) is a psychedelic drug that is produced by psilocybin mushrooms, such as, but not limited to, P. azurescens, P. semilanceata, and P. cyanescens. Psilocin, the active metabolite of psilocybin, inhibits the 5-HT transporter (SERT) whereas LSD stimulates D₁₋₃ receptors but does not interact with the SERT (Rickli et al., 2016). In contrast to LSD, psilocybin and mescaline show no affinity for D2 receptors. The potent dopaminergic receptor agonist properties of LSD have been linked to delayed LSD effects that are possibly distinct from other hallucinogens and possibly more stimulant-like (Mittman et al., 1991; Marona-Lewicka et al., 2005; Marona-Lewicka et al., 2007; Nichols, 2016). LSD and the tryptamines DMT and psilocin are potent agonists at serotonin 5-HT1 receptors while other hallucinogens such as mescaline exhibit low potency at this receptor (Rickli et al., 2016). While no clinical studies have clearly documented a role for the 5-HT1 receptor (Strassman, 1996; Nichols, 2016) in the action of psychedelics, differences between substances may exist. SERT inhibition (Rickli et al., 2016) and increases in serotonin by psilocybin may be associated with greater serotonergic toxicity including nausea and vomiting when psilocybin is used compared to other psychedelics with no interaction with the SERT. Mescaline binds in a similar concentration range to 5-HT_{2A}, 5-HT_{1A} and adrenergic α_{2A} receptors (Rickli et al., 2016).

While the mechanism of the psychedelics with respect to pain is not clear, they can act peripherally and centrally and provide a psychological effect as well as a direct neural effect to treat pain. The psychedelics of the present invention can more rapidly treat pain, provide longer-lasting relief, and provide more pain reduction compared to NSAIDs, opioids and SSRIs. The psychedelics of the present invention can be administered in such a way to treat pain but without hallucinogenic side effects. The psychedelics can also alter the individual's mood to reduce and relieve anxiety that can cause and simultaneously result in pain, in addition to the direct pain reducing effect.

In addition to the administration methods listed below, the psychedelics can also be provided in dosage forms that are more amenable to treating pain, such as, but not limited to, transdermal patches, modified-release oral dosage forms, extended release injection, implanted titration device, intranasal delivery forms, or sublingual delivery forms.

The compounds of the present invention are administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compounds of the present invention can be administered in various ways. It should be noted that they can be administered as the compound and can be administered alone or as an active ingredient in combination with pharmaceutically acceptable carriers, diluents, adjuvants, and vehicles. The compounds can be administered orally, transcutaneously, subcutaneously or parenterally including intravenous, intramuscular, and intranasal administration. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses or a continuous dose over a period of several hours, days, weeks, months, or years.

When administering the compound of the present invention parenterally, it will generally be formulated in a sublingual or buccal dissolving tablet, dissolving film, intranasal powder, intranasal solution, inhaled powder, inhaled solution, transdermal patch, transdermal patch with microneedles or other permeation enhancers, or as a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE 1

An individual is administered a hallucinogenic dose (50-500 μg of LSD) at a fixed level every day for at least 4 weeks for the treatment of a chronic pain condition. Preferably, the individual is treated 8 or 12 weeks or longer. Pain is measured on 100-mm visual analog scale, 11-point numeric rating scale, or 5-point Likert scale.

### EXAMPLE 2

An individual is administered decreasing doses of a psychedelic (100-500 μg of LSD followed by decreasing daily amounts until reaching 100 μg) to treat pain.

### EXAMPLE 3

An individual is administered increasing doses of a psychedelic (10 μg up to 200 μg over the course of several days or weeks) to treat pain.

### EXAMPLE 4

An individual is treated for pain with a psychedelic by administering the psychedelic in the form of a patch, extended release injection, extended release tablet or capsule, or with an implanted device that allows for the titration of doses into the therapeutic range.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method of treating pain, including the steps of:
administering an effective amount of a psychedelic to an individual; and
treating pain in the individual.

2. The method of claim 1, wherein the pain is a type chosen from the group consisting of acute, chronic, nociceptive, neuropathic, inflammatory, and functional.

3. The method of claim 1, wherein the pain is caused by a physical state in the individual's body.

4. The method of claim 1, wherein the pain is caused by an emotional state in the individual's body.

5. The method of claim 1, wherein the psychedelic is chosen from the group consisting of lysergic acid diethylamide (LSD), psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, solvates thereof, isomers thereof, analogs thereof, and homologues thereof.

6. The method of claim 1, wherein said treating step is further defined as providing a psychological effect and a direct neural effect to the individual.

7. The method of claim 1, wherein said administering step is further defined as administering the psychedelic in a form chosen from the group consisting of transdermal patches, modified-release oral dosage forms, extended release injection, implanted titration device, intranasal delivery forms, and sublingual delivery forms.
